(19) United States
(12) Patent Application Publication
(10) Pub. No.: US 2023/0037198 A1
(43) Pub. Date: Feb. 2, 2023

(54) APPARATUSES AND METHODS FOR PRODUCING EMBOLIC PARTICLES WITH ACTIVATED LOADING SITES

(71) Applicant: Varian Medical Systems, Inc., Palo Alto, CA (US)

(72) Inventors: Huali Wang, Austin, TX (US); Theodore Ronald Jackson, Ellicott City, MD (US)

(21) Appl. No.: 17/389,063

(22) Filed: Jul. 29, 2021

(51) Int. Cl. A61L 31/04; A61L 31/16; A61L 31/14; C08J 3/22

(52) U.S. Cl. CPC A61L 31/041; A61L 31/16; A61L 31/145; C08J 3/226; A61K 9/5089; A61L 2300/41; C08J 2329/04; C08J 2333/12

(57) ABSTRACT

A method of producing embolic particles includes forming a master batch of embolic particles, wherein the master batch of particles includes a plurality of negatively charged loading sites. The method also includes modifying the master batch of particles to form an activated batch of particles by reacting the master patch of particles with a bridging agent. The activated batch of particles includes a plurality of activated loading sites configured to bond to a negatively charged drug.

### TECHNICAL FIELD

The disclosure relates to apparatuses and methods for the production of embolic particles with activated loading sites. More specifically, the disclosure relates to apparatuses and methods for the production of embolic particles that include activated loading sites capable of being loaded with negatively charged therapeutic materials such as negatively charged drugs.

### BACKGROUND

Many clinical situations benefit from regulation of the vascular, lymphatic or duct systems by restricting the flow of body fluid or secretions. For example, the technique of embolization involves the introduction of particles into the circulation to occlude blood vessels, for example, so as to either arrest or prevent hemorrhaging or to block blood flow to a structure or organ as a means to restrict necessary oxygen and nutrients to the targeted tissue. Permanent or temporary occlusion of blood vessels is desirable for managing various diseases and conditions.

In a typical embolization procedure, local anesthesia is first given over a common artery. The artery is then percutaneously punctured and a catheter is inserted and fluoroscopically guided into the area of interest. An angiogram is then performed by injecting contrast agent through the catheter. Embolic particles are then deposited through the catheter. Embolic particles have been used, for example, in bland transarterial embolization (TAE) and transarterial chemoembolization (TACE). The goal of TACE or TAE procedures is to controllably embolize a local tumor microenvironment, effectively starving the tumor cells by removing an upstream source of oxygen and glucose.

The embolic particles are chosen, for example, based on the size of the vessel to be occluded, the desired duration of occlusion, and/or the type of disease or condition to be treated, among other factors. Other characteristics are important when choosing an embolic particle such as the material of the particle, coatings of the particle, chemicals that may be delivered or released by the particle and other characteristics. In some examples, drugs can be loaded onto the embolic particles that can then be released after the embolic particles are delivered into a target region.

Current apparatuses and methods of producing embolic particles can result in embolic particles that have particular chemical structures that limit the types of chemicals and/or drugs that can be loaded onto the loading sites of the embolic particles. Such limitation can restrict the types of drugs and other therapeutic agents that can be released during embolic treatment. There exists a need, therefore, for improved embolic particles and related apparatuses and methods of producing such embolic particles that can include loading sites that permit different and/or increased types of chemicals and/or drugs that can be loaded onto the embolic particles to improve the therapeutic options available to medical professionals.

### SUMMARY

The methods and apparatuses described herein are directed to embodiments and examples that can be used to produce embolic particles having activated loading sites that can allow negatively charged chemicals and/or drugs to be loaded onto the embolic particles. The embolic particles of the present disclosure can allow different and/or a wider variety of chemicals and/or drugs to be loaded onto the embolic particles than are currently available using existing embolic particles and/or existing methods and apparatuses. The embolic particles of the present disclosure can be used, for example, to improve therapeutic treatments because drugs previously unavailable for embolic treatments can be incorporated into treatments using the embolic particles of the present disclosure.

In accordance with some embodiments, a method of producing embolic particles is provided. The method can include forming a master batch of embolic particles, wherein the master batch of particles includes a plurality of negatively charged loading sites. The method may also include modifying the master batch of particles to form an activated batch of particles by reacting the master patch of particles with a bridging agent. The activated batch of particles includes a plurality of activated loading sites configured to bond to a negatively charged agent.

In one aspect, the bridging agent may include an ethane - 1,2 -diamine or ethylenediamine solution.

In another aspect, the negatively charged agent may include a nonsteroidal anti-inflammatory drug.

In another aspect, the negatively charged agent may include ketorolac tromethamine.

In another aspect, the method may include loading the activated batch of particles with the negatively charged agent via an ion exchange reaction.

In another aspect, the particles may have a substantially spherical shape having a diameter of about 25 µm to about 125 µm.

In another aspect, the master batch of embolic particles may be formed from a methyl methacrylate (MMA) monomer solution.

In another aspect, the master batch of embolic particles may be formed from a polyvinyl alcohol (PVA) solution.

In accordance with some embodiments, an embolic particle is provided. The embolic particle may include a non-biosorbable hydrogel core and a plurality of activated loading sites configured to bond to negatively charged agents via an ion-exchange based mechanism.

In one aspect, the negatively charged agent may include a nonsteroidal anti-inflammatory drug.

In another aspect, the negatively charged agent may include ketorolac tromethamine.

In another aspect, the particle may have a substantially spherical shape having a diameter of about 25 µm to about 125 µm.

In another aspect, the non-biosorbable hydrogel core may include polymethyl methacrylate (PMMA).

In another aspect, the non-biosorbable hydrogel core may be formed from a polyvinyl alcohol (PVA) solution.

In accordance with some embodiments, an embolic particle is provided. The embolic particle may include a hydrogel core, a plurality of activated loading sites, and a negatively charged drug bonded to the activated loading sites.

In one aspect, the hydrogel core is non-biosorbable.

In another aspect, the hydrogel core may be formed from a methyl methacrylate (MMA) monomer solution.

In another aspect, the hydrogel core may be formed from a polyvinyl alcohol (PVA) solution.

In another aspect, the negatively charged drug may include ketorolac tromethamine.

### BRIEF DESCRIPTION OF THE DRAWINGS

The features and advantages of the present disclosures will be more fully disclosed in, or rendered apparent by the following detailed descriptions of example embodiments. The detailed descriptions of the example embodiments are to be considered together with the accompanying drawings wherein like numbers refer to like parts and further wherein:

FIG. 1 is an illustration of an example ion exchange mechanism that can be used to produce embolic particles loaded with a positively charged drug.

FIG. 2 is an illustration of an example particle activation reaction that can be used to produce activated embolic particles having activated loading sites in accordance with the present disclosure.

FIG. 3 is an illustration of an example ion exchange mechanism that can be used to load a negatively charged drug onto the activated embolic particles of FIG. 2.

FIG. 4 is a flow chart illustrating an example method of producing activated embolic particles in accordance with the present disclosure.

### DETAILED DESCRIPTION

The description of the preferred embodiments is intended to be read in connection with the accompanying drawings, which are to be considered part of the entire written description of these disclosures. While the present disclosure is susceptible to various modifications and alternative forms, specific embodiments are shown by way of example in the drawings and will be described in detail herein. The objectives and advantages of the claimed subject matter will become more apparent from the following detailed description of these exemplary embodiments in connection with the accompanying drawings.

It should be understood, however, that the present disclosure is not intended to be limited to the particular forms disclosed. Rather, the present disclosure covers all modifications, equivalents, and alternatives that fall within the spirit and scope of these exemplary embodiments. The terms “couple,” “coupled,” “operatively coupled,” “operatively connected,” and the like should be broadly understood to refer to connecting devices or components together either mechanically, electrically, wired, wirelessly, or otherwise, such that the connection allows the pertinent devices or components to operate (e.g., communicate) with each other as intended by virtue of that relationship.

In the present disclosure the singular forms “a,” “an,” and “the” include the plural reference, and reference to a particular numerical value includes at least that particular value, unless the context clearly indicates otherwise. When values are expressed as approximations, by use of the antecedent “about,” it will be understood that the particular value forms another embodiment. As used herein, “about X” (where X is a numerical value) preferably refers to ±10% of the recited value, inclusive. For example, the phrase “about 8” preferably refers to a value of 7.2 to 8.8, inclusive. Where present, all ranges are inclusive and combinable. For example, when a range of “1 to 5” is recited, the recited range should be construed as including ranges “1 to 4”, “1 to 3”, “1-2”, “1-2 & 4-5”, “1-3 & 5”, “2-5”, and the like. In addition, when a list of alternatives is positively provided, such listing can be interpreted to mean that any of the alternatives may be excluded, e.g., by a negative limitation in the claims. For example, when a range of “1 to 5” is recited, the recited range may be construed as including situations whereby any of 1, 2, 3, 4, or 5 are negatively excluded; thus, a recitation of “1 to 5” may be construed as “1 and 3-5, but not 2”, or simply “wherein 2 is not included.” It is intended that any component, element, attribute, or step that is positively recited herein may be explicitly excluded in the claims, whether such components, elements, attributes, or steps are listed as alternatives or whether they are recited in isolation.

The embolic particles and the methods and apparatuses related to the production of such embolic particles can allow embolic particles to be produced that include activated loading sites. The embolic particles that include the activated loading sites can then be loaded with negatively charged agents or drugs that can bond to the activated loading sites. In some examples, embolic particles can be produced that initially include negatively charged loading sites. The embolic particles with the negatively charged loading sites can be producing using known and/or existing production methods and apparatuses. The embolic particles with the negatively charged loading sites can undergo a bead activation reaction in which a linker molecule is introduced to the embolic particles. The reaction of the embolic particles with the linker molecules can produce an activated embolic particle that includes a positively charged loading site or other activated loading site that can allow a negatively charged agent or negatively charged drug to be loaded onto the activated embolic particles. This process of producing activated embolic particles allows agents and drugs that were previously unable to be loaded on embolic particles to be loaded onto the embolic particles and used during embolic treatments. With such newly configured embolic particles, new, improved, and/or more effective treatments can be performed that were previously unable to medical professionals.

The particles or microspheres of the present disclosure may be used to treat various diseases and conditions in a variety of subjects. Subjects include vertebrate subjects, particularly humans and various warm-blooded animals, including pets and livestock. As used herein, the term treatment refers to the prevention of a disease or condition, the reduction or elimination of symptoms associated with a disease or condition, or the substantial or complete elimination of a disease or condition. Preferred treatments include embolization treatments.

While the term particle or microsphere is used in the present disclosure, the methods and apparatuses described herein may be used to produce embolic particles that do not have a spherical shape or have shapes and/or dimensions that may vary from a sphere.

The particles or microspheres of the present disclosure can have various sizes that may be desired for a particular procedure or treatment. In some examples, the particles of the present disclosure can be microsphere and can have a diameter in the range of about 5 µm to about 1500 µm. In other examples, the microspheres can have a diameter of about 40 µm to about 1300 µm. For particular applications and/or treatments, the microspheres can have a particular nominal size and can have a particular tolerance. For example, a microsphere used for a particular application or treatment can have a nominal diameter size of about 40 µm and a tolerance of ± 10 µm. In another example, for another application and/or treatment, the microsphere can have a nominal diameter size of about 1300 µm and a tolerance of ± 10 µm. In still other examples, the microspheres can have other nominal ranges and other tolerances in the ranges described above.

In yet other examples, the microspheres of the present disclosure may vary significantly in size, with typical diameters ranging, for example, from 25 µm or less to 5000 µm or more, for example, ranging from 25 µm to 50 µm to 75 µm to 100 µm to 150 µm to 250 µm to 500 µm to 750 µm to 1000 µm to 1500 µm to 2000 µm to 2500 µm to 5000 µm (i.e., including all ranges spanning any two of the preceding values). In some examples the microspheres are substantially spherical with a diameter of about 25 µm to about 125 µm. Where collections of microspheres are measured, at least 95 vol % of the population in the collection may fall within these ranges.

The injectable particles and portions thereof (e.g., cores, coatings, etc.) in accordance with the present disclosure may be formed using a variety of inorganic materials (e.g., glasses, ceramics, metals, etc.), organic materials (e.g., non-polymeric organic compounds, polymers, monomers, etc.), as well as combinations of inorganic and organic materials. In some examples, the microspheres can be made from a methyl methacrylate (MMA) monomer solution. The methyl methacrylate can be polymerized to form cross-linked polymethyl methacrylate (PMMA) microspheres. This step can form the bead of the microsphere from raw solid and liquid chemicals. The input materials that can be used during such polymerization step in the formation of the microspheres can include phosphate buffer saline solution, polyvinyl alcohol solution, lauroyl peroxide, methyl methacrylate, triethylene glycol dimethacrylate, and n-dodecyl mercaptan. MMA monomer droplets can be formed into the PMMA microspheres by depositing the MMA monomer droplets into a polyvinyl alcohol (PVA) solution in which the chemical reaction can take place.

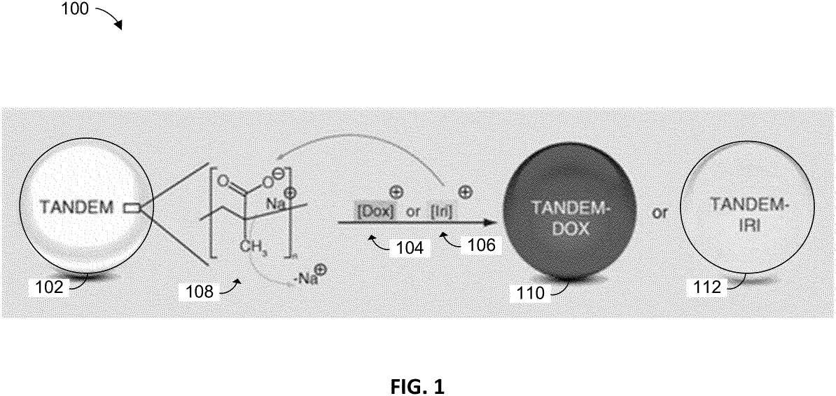

Referring now to FIG. 1, an example drug loading mechanism 100 is shown. In this example, a drug 104, 106 can be loaded via an ion-exchange mechanism onto a master batch embolic particle 102. The master batch embolic particle 102 can be produced using various particle or microsphere production methods known in the art. The master batch embolic particle 102 can include one or more loading sites 108 on the particle. The loading sites 108 can be, for example, a location or feature of the chemical structure of the particle 102 that can allow for the bonding of an agent and/or drug to the particle 102. Such bonding of the agent and/or drug is suitable to cause the agent and/or drug 104, 106 to remain attached and/or coupled to the embolic particle for a suitable bonding duration so that the agent and/or drug 104, 106 can be delivered to a target location in a subject along with the particle 102 during an embolic treatment. The agent and/or drug 104, 106 can then be released at the target location during treatment.

In the example shown, the loading site 108 can be an ion exchange site with a negative charge. The particle 102, therefore, is suitable for the bonding of positively charged drugs 104, 106. After the agents and/or drug 104, 106 are introduced to the master batch particles 102 with the loading sites 108, the drug loading mechanism 100 can result in drug-loaded particles 110, 112.

In the example shown, the master batch particles 102 can be, for example, Tandem® or Oncozene® microspheres offered by Varian Medical Systems, Inc. In such examples, the microspheres 102 can be loaded with various drugs such as doxorubicin (DOX) or irinotecan (IRI). These drugs 104, 106 can be loaded onto the negatively charged polymer backbone of the particles 102 via an ion exchange mechanism, such as mechanism 100. Due to the availability of the loading sites 108 on the polymer structure of the particles 102, the particles 102 have shown an ability to load up to 50 mg of doxorubicin or irinotecan per ml of embolic particles. In other examples, other drugs or agents can be loaded onto the master batch particles 102.

It can be desirable to load other drugs and/or agents onto the master batch particles 102. The loading sites 108, however, can be negatively charged and/or otherwise only capable of loading positively charged drugs. One example drug that can be desirable to load onto the master batch particles is a nonsteroidal anti-inflammatory drug (NSAID) that can be used to treat pain. Such a drug, for example, is provided under the name Toradol® that is also known as ketorolac tromethamine. Ketorolac tromethamine, however, has a negatively charged structure that makes it incompatible for loading onto the loading sites 108 of the master batch particles 102.

In other examples or circumstances, it can be desirable to load other drugs and/or agents onto the master batch particles 102 that may have negatively charged structures that also make such drugs and/or agents incompatible with the master batch particles 102. Examples of such other negatively charged drugs and/or agents include Ketorolac Tromethamine (TORADOL®), Camptothecin, Amphetamine, Phosphoramide Mustard, Cyclophosphamide, etc.

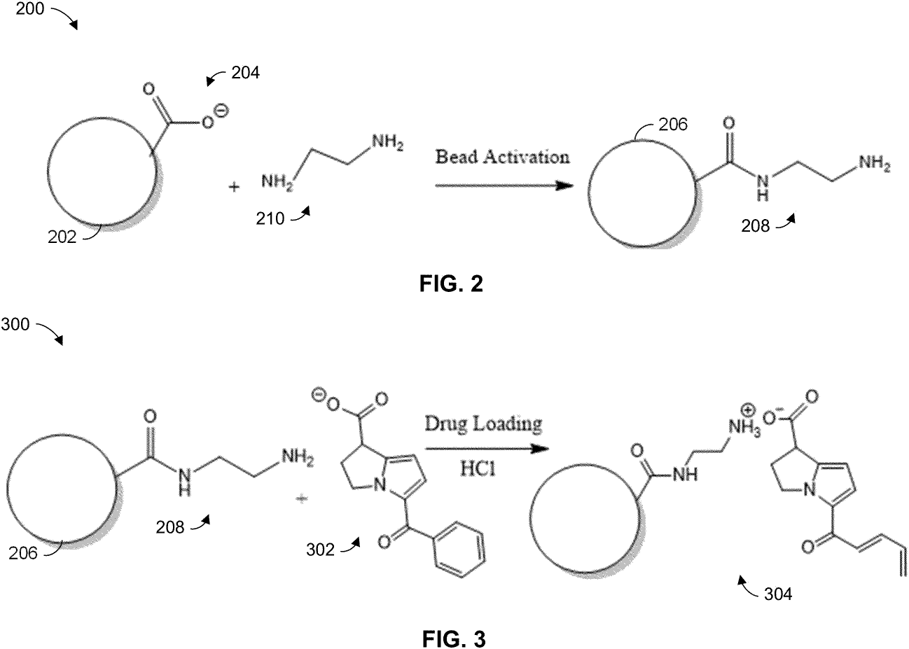

As shown in FIGS. 2-3 and as further described below, the master batch particles can undergo an activation process in which a linker molecule is introduced to the master batch particles to modify the negatively charged loading sites to activated loading sites configured to bond to negatively charged drugs and/or agents. In the examples described below, the particles and methods of the present disclosure are described in an example process in which ketorolac tromethamine is loaded onto the embolic particles. It should be appreciated, however, that the described particles and methods can be used in conjunction with the other negatively charged drugs and/or agents (such as those described above) to load such negatively charged drugs and/or agents to the activated embolic particles.

As shown in FIG. 2, master batch particles 202 can be modified into activated particles 206 using an activation reaction 200 as shown. A master batch particle 202 can be formed similarly as previously described (see FIG. 1) and can include a plurality of negatively charged loading sites 204. In the activation process 200, a linker molecule 210 is introduced to the master batch particles 202. In one example, the master batch particles 202 can be introduced to a solution that includes ethane-1,2-diamine. The diamine can covalently bond to the master batch particle 202 to produce an activated particle 206 that includes one or more activated loading sites 208. The covalent bond can form an activated particle 206 that can have a shelf life of three years or more.

The activated particles 206 include the activated loading sites 208 that are configured to allow negatively charged drugs or other agents to be loaded onto the activated particles 206. In the example shown, the diamine is used as the linker molecule 210. In other examples, other linker molecules can be used such as 1,1-dimethylethylenediamine, 1,3-diaminopropane, butane-1,4-diamine, pentane-1,5-damine, etc.

Referring now to FIG. 3, a process 300 for loading a negatively charged drug is shown. In this example, the activated particles 206 formed in process 200 (FIG. 2) can be used. As previously described, the activated particles 206 can include a plurality of activated loading sites 208. The activated particles 206 can be combined with a solution of a negatively charged drug 302, such as ketorolac tromethamine. The negatively charged drug 302 can bond via an ion exchange mechanism to form an ionic bond to the activated particles 206 to form a drug-loaded embolic particle 304 loaded with the negatively charged drug. Due to the ionic bond between the negatively charged drug and the embolic particle, the shelf life of the drug-loaded embolic particle 304 can be about 48 hours or less.

The process 300 can be performed at a treatment facility, a pharmacy or other location that can be located proximate to the location where the embolic treatment procedure would be performed. Because of the limited shelf life of the drug-loaded embolic particles 304, the process 300 can be performed prior to the embolic treatment at or near the treatment facility. The process 300 can be performed using simple equipment without the need for specialized equipment. The process 300 can be performed, for example, in medical syringes. The negatively charged drug 302 can be mixed with the activated embolic particles 206 in a medical syringe. In other examples, the process 300 can be performed in other containers.

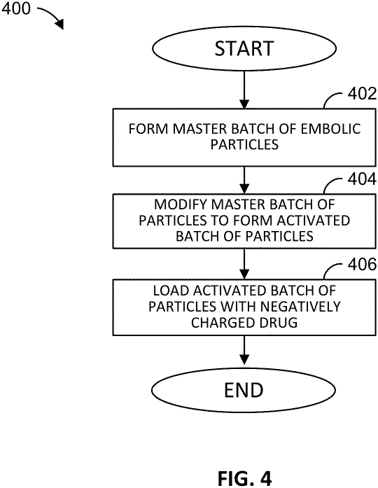

Referring now to FIG. 4, a method 400 of producing embolic particles is shown. The method 400 can be used to produce activated embolic particles that can then be loaded with a negatively charged drug. The method 400 can begin with step 402 in which a master batch of embolic particles is formed. Any suitable process can be used to form the master batch of embolic particles. In one example, the master batch of particles can be formed from a methyl methacrylate (MMA) monomer solution to form polymethyl methacrylate (PMMA) particles or microspheres. In another example, the master batch of particles can be formed from a polyvinyl alcohol (PVA) solution. In other examples, other suitable processes can be used.

As a result of step 402, the master batch of embolic particles have negatively charged loading sites. The master batch of embolic particles can have a structure of the particles 202 shown in FIG. 2. As a result, the master batch of particles are ill-suited to load negatively charged drugs (or other negatively agents).

The method 400 can continue to step 404 in which the master batch of particles can be modified to form an activated batch of embolic particles. The activated batch of embolic particles can, for example, be reacted with a bridging agent to form a plurality of activated loading sites on the activated batch of embolic particles. The activated loading sites can be configured to bond with a negatively charged agent or drug, such as ketorolac tromethamine. In one example, the step 402 can be performed using the reaction as shown in FIG. 2 to modify the particle 202 with the linker molecule 210 to form the activated particle 206. In some examples, the bridging agent can be a solution that includes ethane-1,2-diamine. In other examples, the bridging agent can include other linker molecules.

The method 400 can then continue to step 406. At step 406, the activated batch of particles can be loaded with a negatively charged drug (or other negatively charged agent). The step 406 can be performed, for example, using the process 300 shown in FIG. 3. In such an example, the activated batch of particles can be combined with the negatively charged drug to form drug-loaded embolic particles. The negatively charged drug can be ketorolac tormethamine, for example. In other examples, the negatively charged drug can be a nonsteroidal anti-inflammatory drug. The negatively charged drug can be loaded using an ion exchange mechanism.

The method 400 and the particles and methods of the present disclosure can be used to produce embolic particles, microspheres, microbeads or the like that can include activated loading sites that are configured to allow negatively charged drugs to bond to the activated loading sites. Existing embolic particles do not have suitable structures to allow the loading of negatively charged drugs. The ability to load negatively charged drugs allows medical professionals to perform embolic treatments that otherwise cannot be performed or allows such embolic treatments to have different and/or enhanced performance.

The foregoing is provided for purposes of illustrating, explaining, and describing embodiments of these disclosures. Modifications and adaptations to these embodiments will be apparent to those skilled in the art and may be made without departing from the scope or spirit of these disclosures.

What is claimed is:

1. A method of producing embolic particles comprising:
forming a master batch of embolic particles, wherein the master batch of particles includes a plurality of negatively charged loading sites;
modifying the master batch of particles to form an activated batch of particles by reacting the master patch of particles with a bridging agent, wherein the activated batch of particles includes a plurality of activated loading sites configured to bond to a negatively charged agent.

2. The method of claim 1, wherein the bridging agent comprises a ethane - 1,2 - diamine solution.

3. The method of claim 1, wherein the negatively charged agent comprises a nonsteroidal anti-inflammatory drug.

4. The method of claim 1, wherein the negatively charged agent comprises ketorolac tromethamine.

5. The method of claim 1, further comprising loading the activated batch of particles with the negatively charged agent via an ion exchange reaction.

6. The method of claim 5, wherein the negatively charged agent comprises ketorolac tromethamine.

7. The method of claim 6, wherein the particles comprise a substantially spherical shape having a diameter of about 25 µm to about 125 µm.

8. The method of claim 1, wherein the master batch of embolic particles is formed from a methyl methacrylate (MMA) monomer solution.

9. The method of claim 1, wherein the master batch of embolic particles is formed from a polyvinyl alcohol (PVA) solution.

10. An embolic microparticle comprising:
a non-biosorbable hydrogel core; and
a plurality of activated loading sites configured to bond to negatively charged agents via an ion-exchange based mechanism.

11. The embolic microparticle of claim 10, wherein the negatively charged agent comprises a nonsteroidal anti-inflammatory drug.

12. The embolic microparticle of claim 10, wherein the negatively charged agent comprises ketorolac tromethamine.

13. The embolic microparticle of claim 10, wherein the microparticle comprises a substantially spherical shape having a diameter of about 25 µm to about 125 µm.

14. The embolic microparticle of claim 10 wherein the non-biosorbable hydrogel core comprises polymethyl methacrylate (PMMA).

15. The embolic microparticle of claim 10 wherein the non-biosorbable hydrogel core is formed from a polyvinyl alcohol (PVA) solution.

16. An embolic microparticle comprising:
a hydrogel core;
a plurality of activated loading sites; and
a negatively charged drug bonded to the activated loading sites.

17. The embolic microparticle of claim 16, wherein the hydrogel core is non-biosorbable.

18. The embolic microparticle of claim 16, wherein the hydrogel core is formed from a methyl methacrylate (MMA) monomer solution.

19. The embolic microparticle of claim 16, wherein the hydrogel core is formed from a polyvinyl alcohol (PVA) solution.

20. The embolic microparticle of claim 16, wherein the negatively charged drug comprises ketorolac tromethamine.
